(19) United States
(12) Patent Application Publication
(10) Pub. No.: US 2003/0211036 A1
(43) Pub. Date: Nov. 13, 2003

(54) Method and apparatus for monitoring and quantitatively evaluating tumor perfusion

(76) Inventors: Hadassa Degani, Rehovot (IL); Liora Bogin, Moshav Ganei Yochanan (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/139,993

(22) Filed: May 7, 2002

(51) Int. Cl. A61K 51/00; A61B 5/055; G06F 19/00; G01N 33/48; G01N 33/50; A61K 49/00

(52) U.S. Cl. 424/1.11; 424/9.3; 702/19; 600/410

(57) ABSTRACT

Method and apparatus for monitoring a patient having a tumor to determine perfusion tumor heterogeneity wherein a solution containing a tracer, preferably a ²H-saline solution is infused into the patient's bloodstream at a predetermined slow rate to effect perfusion into the tumor. An MRI machine is adjusted to acquire a set of dynamic ²H magnetic resonance images of the tumor. The ²H-images are obtained before infusion, during infusion and post infusion. First, the obtained images are processed to quantitatively determine perfusion per voxel of the images. Next, maps of perfusion parameters are generated to indicate spatial distribution of tumor perfusion. The maps are displayed in color code and analyzed.

### BACKGROUND OF THE INVENTION

1. Field of the Invention

The present invention relates to a method and apparatus for monitoring and quantitatively evaluating tumor perfusion by monitoring the kinetics of substances comprising small molecules transferable through membranes that can also be tracers, particularly deuterated water with non-invasive imaging method, particularly deuterium MRI, and more particularly, to a method and apparatus for assessing the efficiency of drug delivery to tissue, and a method and apparatus for monitoring response to therapy, especially in the course of anti-angiogenic treatment. The invention also relates to a product of a machine readable medium having stored thereon a novel algorithm for processing dynamic images particularly for calculating the kinetics of tissue perfusion.

2. Prior Art

Growth and development of solid tumors rely on their perfusion that is achieved via complex and tortuous network of capillaries (1-5). The important role of tumor vasculature in tumor growth has made it an appropriate target for anti-cancer therapies (6-8), and efforts have been made to obtain a quantitative measure of tumor perfusion in order to assess the efficiency of distribution of the therapeutic drugs within the tumor, as well as, to evaluate the response to anti-vascular therapy.

Dynamic MRI, which is used for screening and diagnosis of cancer, has been proposed for monitoring perfusion, in-vivo, non-invasively and with an accurate anatomical localization. Studies of human breast cancer tumors by ¹H-contrast-enhanced high resolution MRI, combined with the appropriate pharmaco-kinetic and physiological analysis, have been shown to provide valuable and quantitative mapping of vascular permeability and extracellular space accessible to a contrast agent (9-12). In order to study perfusion, irrespective of a specific permeability, it is most practical to dynamically monitor perfusion of tagged water molecules. A suitable candidate for such application is deuterium-labeled water (HDO) with ²H as the MR detectable and stable isotope. HDO enrichment of body fluids up to 10% is considered nontoxic (13, 14).

Assessment of the average perfusion over a whole tumor or organ by dynamic ²H-MR spectroscopy, with HDO as a tracer, was pioneered by Ackerman and Kim (15). This study laid the groundwork for numerous ²H-NMR investigations with increasing temporal resolution at high signal-to-noise ratio (16-21), however omitting spatial resolution and localization. ²H-MR imaging perfusion studies, initially introduced by Larcombe-McDouall and Evelhoch (22), allowed monitoring the spatial distribution of HDO in tumors, thus, revealing tumor heterogeneous vascularity. A distinct work by Eskey and co-workers mapped quantitatively tumor blood flow per voxel in tissue-isolated rat mammary adenocarcinoma (23).

The kinetic and physiologic parameters that can be extracted from the time course of tracer distribution depend on the theoretical model and its relevant assumptions, used to interpret the data (17, 24). To date, HDO kinetic studies based on Kety's theory (25) and other models, have been aimed to estimate perfusion rate constants, and consequently, tumor blood flow (TBF) but did not account for the volume of the intravascular compartment (16, 17, 21-24, 26-29). The volume fraction occupied by this compartment could be significant, up to 27%, in some tumor tissues (1). Moreover, the magnitude of the intravascular volume fraction may not be directly derived from regional blood flow, as these two physiologic features do not necessarily correlate in tumors (30).

### SUMMARY OF THE INVENTION

The object of the present invention is to provide a method and apparatus for monitoring and qualitatively evaluating tissue perfusion in-vivo using non-invasive imaging methods to follow the uptake and clearance of tracers from the tumor, and more particularly, to a method and apparatus for assessing the efficiency of drug delivery to tissue, and a method and apparatus for monitoring response to therapy, especially in the course of anti-angiogenic treatment.

The tracers disclosed in the present invention belong to the group of small-substances (molecules), transferable through membranes, that can be tracers which are detectable, non-toxic at the concentrations required for detection, that flow in blood with a rate similar to the blood flow, including but not limited to water and water labeled with tritium, or ¹⁷O labeled water (H₂¹⁷O), or ¹⁸O labeled water, sugars including mannitol and sugars including mannitol labeled with ¹⁴C, ¹³C or ²H or ³H or ¹⁷O or ¹⁸O, alcohols including ethanol and alcohols labeled with ¹⁴C, ¹³C or ²H or ³H or ¹⁷O or ¹⁸O, organic acids including acetic and lactic acid and organic acids with ¹⁴C, ¹³C or ²H or ³H or ¹⁷O or ¹⁸O, amines including ethanolamine amino acids and analogs of amino acids and amines with ¹⁵N, ¹⁴C, ¹³C or ²H or ³H or ¹⁷O or ¹⁸O and small fluorinated compounds with either ¹⁹F or ¹⁸F. The preferred substance is deuterated labeled water, also termed hereinafter ²H₂O or HDO.

The object of the present invention is to provide a method and apparatus for monitoring the uptake and clearance of tracers, particularly deuterated water, following a quantitative evaluation of tissue perfusion, particularly tumor perfusion, with MRI, and especially deuterium MRI, and more particularly, to a method and apparatus for assessing the efficiency of drug delivery to tissue, and a method and apparatus for monitoring response to therapy, especially in the course of anti-angiogenic treatment.

The objects of the present invention are generally illustrated for ²H-MRI. However, the invention is equally applicable to other imaging techniques, relying on other tracers.

Although microcirculation of solid tumors is known to be heterogeneous due to the abnormal architecture and morphology of tumor vasculature (2, 3), the results obtained to date are deficient with respect to accuracy. The aim of the present invention is to provide a method and apparatus that more accurately monitors and quantitatively evaluates the perfusion, with its heterogeneity, in tissues including but not limited to solid tumors, and particularly in breast cancer tumors. This is accomplished by carrying out the method and operating the apparatus in a manner such that the spatial resolution is substantially increase and the signal-to-noise ratio (SNR) of the dynamic ²H MR-images and processing the acquired data with the same resolution as it was acquired. Optimization of the protocol of the invention, for the purpose of high SNR, was achieved by image acquisition with 3D sequence and enrichment of the body with high, non-toxic, levels of HDO by a slow i.v. infusion. The resulting concentration of HDO in the plasma upon infusion was ˜6 times higher with respect to bolus injection of 200 μl of this tracer.

Recently, the critical role of high spatial resolution in MRI model-based diagnosis of breast tumors was demonstrated (44). Most in-vivo perfusion studies to date have favored the time resolution on the account of the spatial resolution. As a test regarding the efficacy of the invention, by applying the kinetic model-based analysis on the ²H-dynamic data with a degraded spatial resolution (by a factor of 8), it was shown that extreme values of perfusion parameters along with the spatial heterogeneity and distribution of these parameters were no longer detectable. The perfusion parameters obtained from the spatially degraded images were found to be similar to their average values that were calculated from the highly resolved maps. However, the later values were shown to poorly represent the actual data due to their asymmetric distribution. Thus, it is significant to the present invention that the method not lower the resolution, nor acquire the data globally over the whole tumor for to do so will yield values that do not represent statistically the actual perfusion parameters.

Obtaining kinetic parameters from the dynamic data, according to the kinetic model of the present invention, requires knowledge of the arterial concentration of HDO, namely the arterial input function (AIF). Recently a methodology to overcome the difficulty in reproducing the AIF was introduced, which involves surgical implantation of catheters within the carotid artery (21, 45). These studies concluded that once AIF is determined for a given set of animals it could be used for perfusion estimation of other sets of animals with similar animal model and experimental protocol. In the experimental and testing phases of the present invention, the AIF was determined in a group of mice, remotely of the magnet, and the results applied within the model-based analysis of ²H MR dynamic images that were measured in different mice, but with the same HDO dose and infusion protocol. The AIF was determined by blood withdrawal, as this type of measurement was deemed more practical for clinical application. Also, the invention uses the intuitive assumption that mixing of HDO in the heart results in a homogeneous arterial tracer concentration, and thus, sampling arterial blood from any major vessel is representative of the arterial blood concentration and moreover of the supply to the tissue of interest, i.e. the tumor. The results obtained by the invention were reproducible with a sampling time of 15 s that was high with respect to the sampling time of the ²H-MRI images (2.05 min).

Tumor vasculature was shown to be relatively permeable by contrast enhance MRI studies (46, 47), and by photometric and microscopic analyses (48, 49). It is, therefore, commonly assumed that flow is rate limiting in the process of HDO perfusion. However, it was discovered by the present invention that the time evolution of HDO perfusion was not parallel to that of HDO in the plasma within many voxels with high intravascular volume fraction leading to the conclusion that in addition to the contribution of flow, vascular permeability also contributed to the magnitude of HDO perfusion (see Eq. [3]). This discovery does not contradict common knowledge regarding vascular permeability, but rather contributes to deeper understanding of the perfusion concept in tumors. Specifically, the chaotic nature of tumor vasculature may result in flow rates that are of the same order as the permeability and surface area product. Thus, in confined tumor loci, flow will not be exclusively rate limiting. Conversely, the abnormal architecture of tumor vasculature (1-5) may locally mask permeability due to capillary tortuosity. Thus, the advantage of HDO as a tracer in perfusion technique, over macromolecules, is that it allows observing voxels in which flow is rate limiting, as well as, voxels in which contribution from capillary permeability affects the process of perfusion. Further evidence to the irregularity of tumor vasculature came from analysis of the parametric maps and the finding that the perfusion rate constants did not correlate with the intravascular volume fraction, per voxel. This finding strongly indicated that the net quality of perfusion was not necessarily related to the extent of vascularity in the tumor tissue as was shown in normal tissue (30).

A comparison of the perfusion rate K obtained by the present invention with tumor blood flow values from the literature (16, 17, 21-23, 26-29) could not be directly applied as it required adjustment of the units. The sensitivity of the protocol, in addition to the analysis at high spatial resolution, enabled detection and evaluation of the perfusion heterogeneity. Particularly, the inventive methodology allowed the determination of the ‘hot spots’ of perfusion, namely, loci that are highly perfused, as well as, evaluation of perfusion in voxels that are poorly perfused. The sensitivity of measurements made by the method and apparatus of the present invention was further confirmed by comparison of the maps and data with similar analysis at degraded resolution (FIG. 6), as already mentioned.

Evaluation of the intravascular volume fraction, vₚ, in parallel to that of the perfusion rate constant was omitted from most perfusion studies, assuming that it is low and thus negligible. Vascular space determined in Clouser human breast carcinoma grown in athymic mice was found to be ˜4% (1). Yeung et al. have shown that the average vₚ in human brain tumors is 0.068±0.011 ml·g⁻¹ with a corresponding kₜ of 0.0273±0.006 ml·g⁻¹·min⁻¹. The kₜ values found by that study are within the range of TBF values found in rat gliosarcoma by dynamic ²H-MRS (21). Thus, in the practice of the invention, vₚ was evaluated for each voxel in parallel to the evaluation of K or k^{t} aiming to verify its significance. The results obtained show that the range of vₚ of 0.4% to 35% obtained from the total number of voxels in the seven MCF7 tumors, is in agreement with the literature. Moreover, the average value of vₚ (14.2%±0.2%) leads to the inventive conclusion that this parameter must be included in perfusion analysis.

The heterogeneity of the perfusion parameters in MCF7 human breast cancer highlights the importance of carrying out the inventive method and operating the inventive apparatus for measuring and processing perfusion data at high spatial resolution. In addition the measurable magnitude of the intravascular volume fraction in most voxels requires that this parameter be evaluated in parallel to the evaluation of the perfusion rate constant. Finally, the methodology of the present invention is useful in the ancillary method and apparatus that utilizes the capability of MRI as a non-invasive imaging tool for assessing the efficiency of drug delivery to tissue. Further, a novel method and apparatus is disclosed herein that can be used for monitoring response to therapy, particularly in the course of anti-angiogenic treatment.

Thus, the object achieved by the present invention was to quantitatively evaluate the intravascular volume fraction in parallel to the perfusion rate constants, per voxel, in tumors, and more particularly, in MCF7 human breast cancer tumors. The invention was experimentally tested using MCF7 human breast cancer tumors implanted in the mammary fat pad of CD1-NU mice. To this end the invention includes the development of a procedure that allows acquisition of dynamic ²H-MR images with high spatial resolution at adequate signal-to-noise ratio (SNR). Further, the invention includes the development of computational processing algorithms based on Patlak's 2-compartment kinetic model (31, 32). Analysis of the dynamic data yielded maps of the perfusion parameters and revealed their spatial heterogeneity.

Other and further object and advantages of the present invention will become readily apparent from the following detailed description of the inventive method and apparatus when taken in conjunction with the appended drawings.

### BRIEF DESCRIPTION OF THE DRAWINGS

FIG. 1 shows HDO time course during and post ²H-saline infusion, with FIG. 1a showing the concentrations of HDO in mice plasma, given as mean±SEM (n=3). Rates of HDO accumulation during ²H-saline infusion and clearance post-infusion are represented by the linear functions f(t₀−t_{infus}) and g(t_{infus}−t_{end}), respectively (dashed lines). FIG. 1b shows selected ²H MR images acquired pre-infusion (0 min), during infusion (3, 5 and 9 min) and post-infusion (15, 20, 30 and 40 min). The complete series contained 20 images with a time resolution of 2.05 min and a spatial resolution of 0.0052 cm³.

FIG. 2 shows parametric maps of HDO perfusion, the maps (panels a to d) showing perfusion K, intravascular volume fraction-vₚ and a proportion of variability-R² were calculated by the model-based analysis. The maps represent 2 slices of a small tumor, 0.54 cm³ (panels a and b) and a large tumor, 1.50 cm³ (panels c and d). The maps shown in panel a represent the analysis of the tumor shown in FIG. 1 in the images marked b. The digital range of each parameter is coded according to the color scaling shown at the bottom of the maps, red being at the right end and dark blue at the left end, as shown. The boundaries of the tumors are outlined in white.

FIG. 3 shows typical time course profiles of HDO concentration. The resulted fit to the HDO perfusion kinetic model in 4 voxels is as follows: (a) K=1.6×10⁻³ min⁻¹, vₚ=31.9% and R²=0.94; (b) K=5.0×10⁻⁴ min⁻¹, vₚ=31% and R²=0.94; (c) K=3.1×10⁻³ min⁻¹, vₚ=13.5%, and R²=0.96; and (d) could not be fitted, R²˜0.

FIG. 4 shows perfusions rate versus intravascular volume fraction in 4 slices from 4 different tumors: A. 1.5 cm³; B. 0.54 cm³; C. 0.27 cm³; D. 1.79 cm³. Each data point presents K and vₚ of a single voxel.

FIG. 5 shows frequency distribution of K and vₚ in 3 representative tumors (a-c), with bin size being 2·10⁻⁴ min⁻¹ for K and 1.5·10⁻² for vₚ. The star, in each histogram, indicates the position of the mean value to the given data.

FIG. 6 shows parametric maps at the original high resolution (left panels) and at the in-plane degraded resolution (right panel) of a selected tumor. Tumor's region of interest (ROI) is outlined in white. The borders of each pixel at the spatially degraded maps, which represents 64 pixels in the high-resolution maps, are marked in white in the former maps. The white grid in the high resolution maps represents the pixels in the corresponding degraded-resolution maps. The bar in the lower left corner of R² map at high resolution represents the length scale.

FIG. 7 is a flowchart showing the apparatus of the invention.

### DETAILED DESCRIPTION OF PREFERRED SPECIFIC EMBODIMENTS

The method and apparatus of the present invention will now be described in detail with respect to preferred embodiments. The novel method and apparatus is directed to monitoring and quantitatively evaluating tumor perfusion by the steps of monitoring the kinetics of substances comprising small molecules transferable through membranes that can also be tracers, particularly deuterated water with non-invasive imaging method, particularly deuterium MRI, processing the data obtained from the scanning by a unique algorithm, and presenting the processed and analyzed data in the form of parametric maps or images that are color coded. The method and apparatus is also directed to a system for assessing the efficiency of drug delivery to tissue, and to a system for monitoring response to therapy, especially in the course of anti-angiogenic treatment.

Discussing first the method, the invention provides a method for monitoring tissue perfusion comprising the steps of enriching a living tissue mass, which may including a tumor, with a substance characterized by or having the properties of being detectable by whatever scanning technique is being employed, being non-toxic at the concentrations required for detection to the tissue mass, and having a flow rate similar or faster than that of the blood flow; then, monitoring the tracer concentration in the tissue before enrichment, during enrichment and post enrichment by an imaging technique, which can be selected from one of MRI, optical imaging, computed tomography (CT), ultrasound or positron emission tomography (PET), to obtain dynamic images, essentially in digital form; then, processing the obtained images to quantitatively determining perfusion parameters per voxel of the tissue; and finally, obtaining maps of the perfusion parameters to indicate spatial distribution of the parameters and accordingly the tissue perfusion. The maps are then displayed or printed or otherwise put into visual form to be capable of inspection and analysis by a suitable professional.

In the case of application of the method to assessment of drug delivery, the method comprises the steps of performing the above described method using a tracer with the drug and monitoring the tracer concentration to determine the perfusion per voxel to obtain an indication of the efficiency of the drug delivery. Similarly, the method as applied to monitoring response to therapy during a course of anti-angiogenic treatment uses a tracer during the therapy and monitors the tracer concentration to determine the perfusion per voxel to obtain an indication of the response to the therapy in a single treatment, and, particularly, when over a course of treatment in order to compare the response to the therapy over the course of an anti-angiogenic treatment.

The tracer that can be used in the method is a tracer that can be selected from one of the following: water and water labeled with deuterium, or tritium, or ¹⁷O labeled water (H₂ ¹⁷O), or ¹⁸O labeled water; sugars including mannitol and sugars including mannitol labeled with ¹⁴C, ¹³C or ²H or ³H or ¹⁷O or ¹⁸O; alcohols including ethanol and alcohols labeled with ¹⁴C, ¹³C or ²H or ³H or ¹⁷O or ¹⁸O, organic acids including acetic and lactic acid and organic acids with ¹⁴C, ¹³C or ²H or ³H or ¹⁷O or ¹⁸O; amines including ethanolamine, amino acids and analogs of amino acids and amines with ¹⁵N, ¹⁴C, ¹³C or ²H or ³H or ¹⁷O or ¹⁸O; and small fluorinated compounds with either ¹⁹F or ¹⁸F; glucose labeled with ¹³C or ²H or ³H. The preferred tracer is a water labeled with deuterium, and the preferred enrichment is carried out using deuterated-saline solution. Preferably the enriching step is carried out by infusing into the living tissue mass containing blood capillaries a deuterated-saline solution to obtain high and non-toxic levels of HDO in the blood.

The perfusion parameters that are of significance in the method include intravascular volume fraction of the tissue, perfusion rate constants or perfusion rate. Also, there is a further step in the method of generating a proportion of variability map that is of value. According to the method, the images are processed at voxel resolution wherein the voxel size is less than about 0.01 cm³.

The processing step in the method is carried out using a pair of equations, one for time 0 to end of infusion and one for time end of infusion to end of magnetic resonance imaging, and best fitting obtained image data at a voxel resolution wherein the best fit is greater than 0.7 in a major fraction of the voxels. Thereafter, a proportion of variability map is generated. All the generated maps are portrayed in color code.

The method of the present invention is further directed to monitoring a patient having a tumor to determine perfusion tumor heterogeneity comprising the steps of infusing into the patient via the patient's bloodstream a substance characterized by being detectable, non-toxic at the concentrations required for detection, and having a flow rate similar (substantially equal to) or faster than that of the blood flow; monitoring the tumor before infusion, during infusion and post infusion to obtain dynamic ²H magnetic resonance images with predetermined high spatial resolution; processing the obtained images to quantitatively determine perfusion per voxel of the images; and obtaining maps of the perfusion parameters to indicate spatial distribution of the tumor perfusion. The mapped perfusion parameters include intravascular volume fraction of the tissue, perfusion rate constants or perfusion rate. The substance infused is a tracer selected from the tracers mentioned above. The preferred tracer or infusion substance for enrichment is deuterated-saline solution. The enriching step of the method is carried out, preferably, by intravenous infusion of a deuterated-saline solution to obtain high and non-toxic levels of HDO in the blood. The processing step of the method can be carried out so that the images are processed at voxel resolution wherein the voxel size is less than about 0.01 cm³. Also, a proportion of variability map can be generated.

In the method as described above, the processing step is carried out using a pair of equations, one for time 0 to end of infusion and one for time end of infusion to end of magnetic resonance imaging, and best fitting obtained image data at a voxel resolution, wherein the best fit is greater than 0.7 in a major fraction of the voxels.

The generated maps are portrayed in color code, and the generated maps are displayed.

The present invention will be illustrated by a detailed description of the preferred embodiments, which are deuterated water and deuterium MRI that are considered to be the best modes for implementing the invention, and that includes a detailed description of the novel algorithm of the invention.

The method and apparatus of the present invention will now be described in detail with reference to the appended drawings. The process of tissue perfusion in accordance with the present invention can be monitored by introducing a labeled tracer to the blood circulation and following its delivery and clearance in the tissues of interest. The tracer, injected intravenously, circulates within the global blood system and diffuses from the blood to the tissues through capillary wall that are permeable to the tracer molecule. Thus, tissue perfusion is determined by three factors: global blood flow, the volume of capillaries occupying the tissue and capillary permeability to the tracer. The extravascular compartment includes the cellular compartment, however, water diffusion across the cell membrane is ignored due to its fast exchange between the intracellular and the extracellular compartments. The rate constant of this exchange ranges between 100-600 min⁻¹ (37-39), which is much higher than the rate constants of HDO perfusion (17, 28). The principal purpose of the invention was to quantitatively evaluate the spatial distribution of perfusion in MCF7 human breast cancer tumors implanted in the mammary fat pad of nude mice as a proof of principle for the inventive method and apparatus. Deuterated water was shown to be a proper tracer for perfusion studies due to its availability and low toxicity (40). Another important property of this tracer is that its perfusion is mainly determined by flow and is less dependent on capillary permeability (20).

A two-compartment model was custom developed for this activity to describe the kinetics of HDO in MCF7 tumors. The model, designed by Yeung et al. (41) to calculate blood brain transfer constant, is a special case of Patlak's blood-brain exchange model (31, 32). In the present work, the two compartments were assigned to the intravascular space with its respective volume fraction vₚ and the extravascular space, with its effective volume fraction of water vₑ* which is approximately 70% of the actual extravascular volume vₑ (vₑ*=0.7 vₑ) their respective volume fraction vₚ and vₑ* where,

vₚ+vₑ*=I [I]

The rate of HDO perfusion K, the rate constant of the transfer between vₚ to vₑ* (kₜ) and the back-flux rate constant from vₑ to vₚ (k_{b}) are related as follows:

K=vₚkₜ=vₑ*k_{b} [2]

The magnitude of these rate constants is determined by the blood flow (f) and permeability×capillary surface area (PS) according to:

$${k}_{b} = \frac{f ( \frac{1 - {e}^{- PS}}{f} )}{{Ve}^{*}} \qquad [ 3 ]$$

It should be noted that in practice f and PS refer to the net values of these parameters in a given voxel rather than to a unidirectional flow at a single capillary and permeability of a specific capillary. Equation [3] indicates that in the case where tracer's perfusion is not limited by the capillary permeability and surface area product, PS, then the expression: 1−e^{-PS/f}, also known as the extraction fraction, converges to unity and thus k_{b}·vₑ*≈f. In the opposite case, when PS is the limiting process, i.e. PS<<f, then the extraction fraction converges to PS/f and thus k_{b}·,vₑ* ≈PS. In the instance of water, the former assumption might be more relevant than the later, however, in light of the abnormal architecture of tumor vasculature, it is also possible that both processes will proceed with similar rates. By conservation of the mass, the change of the concentration of HDO, Cₑ, in the extravascular volume fraction, vₑ can be expressed as:

$$\frac{ⅆ Ce ( t )}{ⅆ t} = {k}_{t} {C}_{p} ( t ) - {k}_{b} {C}_{e} ( t ) \qquad [ 4 ]$$

where Cₚ(t) is the concentration of HDO in the intravascular compartment and is also the arterial concentration. The solution for Eq. [4], with the initial conditions that at t=0: Cₑ(t)=Cₚ(t)=0 and that upon injection of HDO Cₚ(t) varies with time, is:

$${C}_{e} ( t ) = {k}_{b} {\int }_{0}^{t} {C}_{p} ( T ) {ⅇ}^{- {k}_{b} ( t - T )} ⅆ t \qquad [ 5 ]$$

Voxels sampled by MRI include both compartments and hence the amount of tracer measured by MRI in a voxel, Cᵥ, is given by the following expression:

$${C}_{v} ( t ) = {v}_{e} * {C}_{e} ( t ) + {v}_{p} {C}_{p} ( t ) = {v}_{e} * {k}_{b} {\int }_{0}^{t} {C}_{p} ( T ) {ⅇ}^{- {k}_{b} ( t - T )} ⅆ T + {v}_{p} {C}_{p} ( t ) \qquad [ 6 ]$$

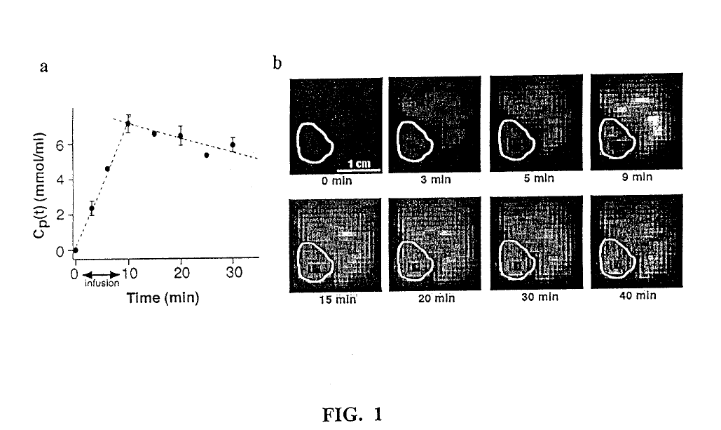

The solution of Eq. [6] requires knowledge of Cₚ(t). In this study Cₚ(t) was determined empirically (FIG. 1a). Thus, the solution for Eq. [6] has two parts: (i) during ²H-saline infusion (t=0−t_{infus}) and (ii) post-infusion (t=t_{infus}−t_{end})- In the first part, during infusion, the expression for the clearance,

$${ⅇ}^{- {k}_{b}^{t}}$$

was replaced by

$$1 - {ⅇ}^{- {k}_{b}^{t}}$$

(42) and Cₚ(t) was given by f(t)=at+b resulting in the following solution for Eq. [6]:

$${C}_{v} ( t ) = {v}_{e} * ( a \cdot t - ( \frac{a}{{k}_{b}} - b ) ( 1 - {ⅇ}^{- {k}_{b}^{t}} ) ) + f ( t ) {v}_{p} = {v}_{e}^{*} \cdot {C}_{e} ( {t}_{infus} ) + f ( t ) {v}_{p} \qquad [ 7 ]$$

where t=0−t_{infus}

Post-infusion Cₚ(t) was given by g(t)=ct+d and hence the solution of Eq. [6] is given by:

$${C}_{v} ( t ) = {v}_{e} * ( c \cdot ( {t}^{′} \cdot {e}^{- {k}_{b}^{{t}^{′}}} ) - ( \frac{c}{{k}_{b}} - d ) ( 1 - {e}^{- {k}_{b}^{{t}^{′}}} ) + {C}_{e} ( {t}_{infus} ) {e}^{- {k}_{b}^{{t}^{′}}} ) = g ( t ) {v}_{p} \qquad [ 8 ]$$

where t′=t_{infuse}−t_{end}

The experimental kinetic data were fitted to Eq. [7] and Eq. [8] with two free parameters: K and vₚ, using the following substitutions: vₑ*=1−vₚ and k_{b}=K/(1−vₚ). Deuterium signal intensity per pixel in arbitrary units was converted to Cᵥ(t) in mmol/ml using a calibration solution of known deuterium concentration in saline solution ( 9.9 mmol/ml).

The method and apparatus of the present invention were tested experimentally according to the following protocol. Female CD1-NU athymic mice, 6 weeks old, were orthotopically inoculated in the mammary fat pad with MCF7 human breast cancer cells as previously described (33). Tumors were allowed to develop for 4-8 weeks, to sizes ranging between 0.2-2 cm³ with average size 0.8±0.2 cm³. For the MRI measurements, mice were anesthetized by exposure to 1% isoflurane (Medeva Pharmaceuticals, Inc., Rochester, N.Y.), in an O₂/N₂O (3:7) mixture, applied through a nose cone. Approval was obtained for all animal procedures according to the guidelines of the Committee on Animals of the Weizmann Institute of Science.

Enrichment of the mice's blood with HDO (99.8%) was achieved by applying a slow i.v. infusion of I ml ²H-saline with a rate of 100 μl/min. The infusion protocol enabled high, non-toxic, levels of HDO in the blood and had no adverse effects on the general well-being of the animals.

Blood samples (˜I ml) were drawn from anesthetized mice by retro orbital sinus puncture at 7 time points, 3 mice for each time point, during and post ²H saline infusion. Samples were collected into separate tubes containing heparin (50 units/ml of blood, Elkins-Sinn Inc., Cherry Hill, N.J.) and were centrifuged at room temperature. The plasma supernatant samples were separated and transferred into 5 mm NMR tubes. ²H-MR spectra of the plasma samples were recorded at 61.4 MHz on DMX-400 spectrometer (Bruker Analytik, Germany), with a broad-band probe by applying 90° pulses with 4 s repetition time (fully relaxed conditions). Choline-(CD₃)₃ was added to each plasma sample as an internal reference for concentration.

In-vivo imaging was accomplished by taking MR images and recording with a 4.7 T Biospec spectrometer (Bruker Analytik, Germany). ²H and ¹H images were recorded at frequencies of 30.7 MHz and 200 MHz, respectively, and detected by a home-built double-tuned ²H/¹H surface coil system, 1.5/2.5 cm in diameter. Dynamic ²H MR images were acquired utilizing a 3-dimensional (3D) gradient echo sequence with the following parameters: echo time (TE)=3 msec, inversion time (TR)=60 msec, flip angle=45° achieved by an adiabatic pulse (sin/cos), 2 averages, matrix—128×64×16, 2.6 mm slice thickness, an in-plane resolution of 1×2 mm₂ and acquisition time of 2.05 min. The sequential acquisition of ²H-images began with a pre-infusion image and continued throughout ²H-saline iv. infusion, into the tail vein of the mouse (1 ml/10 min.), and for 30 minutes thereafter. The pre-infusion images were recorded with a tube containing 9.9 mmol/ml HDO in saline solution, attached to the tumor. This served to calibrate the signal intensity to deuterium concentration in units of mmol/ml.

¹H-T₂-weighted Rapid-Acquisition with Relaxation-Enhancement (RARE) spin-echo (34) multi-slice images were acquired, prior to the dynamic ²H-MRI, with TE/TR of 73/4000 msec at two spatial resolutions: 0.39×0.39×1 min and 1×2×2.6 mm³.

Image analysis was applied on the time evolution of ²H intensity in a series of coronal images that were reconstructed from dynamic 3D images of the whole tumor acquired before, during and post ²H-saline infusion. Analysis was performed utilizing a nonlinear least-square fitting algorithm, developed for the purpose of the invention, at a voxel resolution. The computed analysis initially converted ²H intensity to concentration by referring to the intensity at the pre-infusion image (t=0) in the attached calibration tube that contained a solution of HDO at a concentration of 9.9 mmol/ml. The output of the model-based algorithm, for each voxel in a series of ²H images, was the perfusion rate map K in min⁻¹, and the intravascular volume fraction map (vₚ) and a proportion of variability map (R²) which reflects the quality of the fitting (35). These parameters were color-coded, with the highest values assigned red and the lowest ones assigned black, and were displayed as parametric maps for each tumor slice.

The effect of spatial resolution was examined by performing image analysis at descending pixel resolutions. An automated program averaged the intensities in adjacent pixels in the original MR images to create lower-resolution images. This procedure is not the same as acquiring the images at reduced spatial resolution in terms of SNR. In the artificial reduced spatial resolution images, the SNR is lower by 2^{3/2} than the SNR in images initially acquired at such resolution (36), however, the relative changes remained the same. The spatially degraded images (by a factor of 8) were processed as described above, according to the model-based algorithm. Statistical analysis, including frequency histograms and Student t-test, was processed using Microsoft© Excel and Microcal™ Origin® softwares.

Sectioning the tumors for obtaining a histology plane was established by a single cut through the center of the tumor at the orientation that was used during the MRI study. The two sections of the tumor were then fixed in 2.5% fresh formaldehyde solution and embedded in paraffin. Representative ˜5 μm thick slices from each section were stained with Hematoxylin-Eosin (H&E) to provide a comparison of the MR images to histopathology.

The results obtained for deuterium in the plasma were as follows. The 2-compartment kinetic model related the concentration of the tracer in the tissue to tissue's perfusion, as shown by Eq. [3]. Thus, knowledge of the changing arterial tracer concentration over time, termed arterial input function, was required for the numerical solution of the model. In order to obtain qualitative ²H-images of the tumors and to allow monitoring of HDO uptake with high spatial resolution, a slow i.v. infusion of ²H-saline, 1 ml in 10 min. was applied. This protocol enriched the content of HDO in the plasma, which is 0.0148% at natural abundance, by 6.8% of HDO that is below toxic levels (13, 14). HDO concentration in the plasma, determined by ²H-MRS, was monitored during and post-²H saline infusion (FIG. 1A). During the infusion, HDO accumulated linearly with a correlation coefficient, R², of 0.996. The slow decay of HDO in the plasma, post-infusion, approximated linearity with R²=0.730. Thus, a linear regression analysis of the blood input function Cₚ(t), was applied in parts, producing two linear functions: (i) during ²H-saline infusion at t=t₀−t_{infus}, Cₚ(t)=f(t)=at+b and (ii) post ²H-saline infusion at t=t_{infus}−t_{end}, Cₚ(t)=g(t)=ct+d. The rates of HDO accumulation during infusion and clearance post infusion were a=0.67 mmol·ml⁻¹·min⁻¹ and c=−0.07 mmol·ml^{-1·}min⁻¹, respectively. The plasma concentrations of HDO before and at the end of ²H-saline infusion were b=0.016 mmol·ml⁻¹ and d=7.69 mmol·ml⁻¹, respectively.

Dynamic 3-dimensional ²H-MR images of the whole volume of seven MCF7 breast cancer tumors, implanted in the mammary fat pad of CD1-NU mice, were acquired before, during and post i.v. infusion of ²H-saline. The time course of HDO concentration within slices of the tumors, at a voxel resolution of 0.0052 cm⁻³, was monitored in 2D coronal images that were reconstructed from the 3D images of the tumors with a 2.6 mm slice thickness (FIG. 1b). In those images the signal intensity was directly related to HDO concentrations.

Parametric maps were constructed as follows. Analysis of the HDO time evolution according to algorithms based on Eq. [7] and Eq.[8] yielded the following parameters: intravascular volume fraction—vₚ and its related extravascular volume fraction—vₑ* (Eq. [I]), a perfusion rate K ( min⁻¹) and its related transfer rate constant—kₜ (min⁻¹; Eq. [2]). In addition, a proportion of variability parameter—R², which describes the quality of the fit (35), was assigned to each voxel. The two independent parameters, vₚ and K, were calculated in two steps, once from the HDO uptake phase that was monitored during the infusion, and secondly, from HDO clearance phase post the infusion. The computed analysis however was restricted to yield for each voxel the values that were identical in both phases and were of best fit (high R² value) to the model.

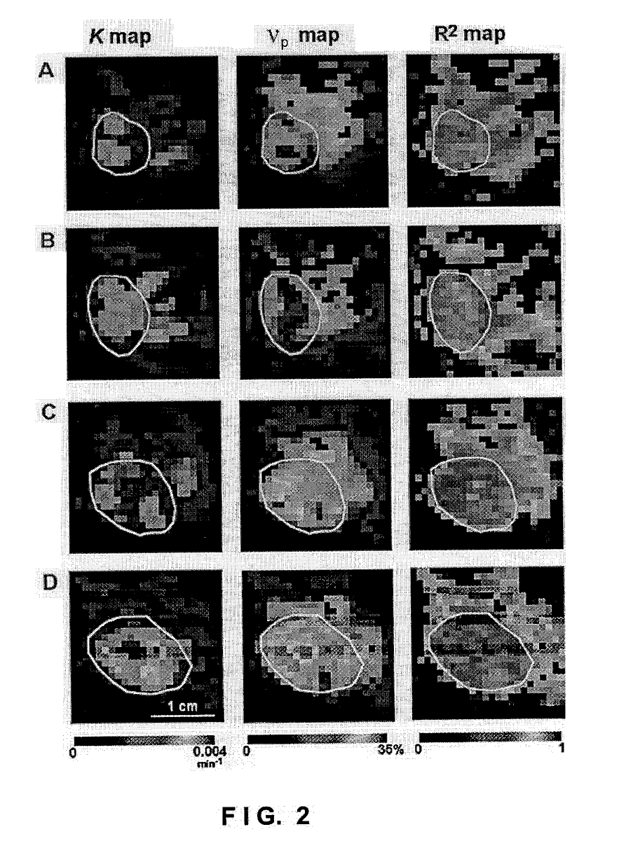

Maps of the perfusion rate K and the intravascular volume fraction—vₚ reflected the spatial distribution of these parameters in the tumors (FIG. 2). The maps also revealed the heterogeneity of the parameters from one tumor to the other and within slices of the same tumor. The perfusion parameters were of a high quality of fit, R²>0.7, in a major fraction (95%) of the voxels that were analyzed in all the tumor slices (n=13). The spatial heterogeneity of K, and particularly of vₚ, within the tumor slices, was related to the bulk morphological feature of the tumors. Clearly, voxels within necrotic loci did not fit to the kinetic model, and thus, were not included in the statistics and were assigned black in the parametric maps.

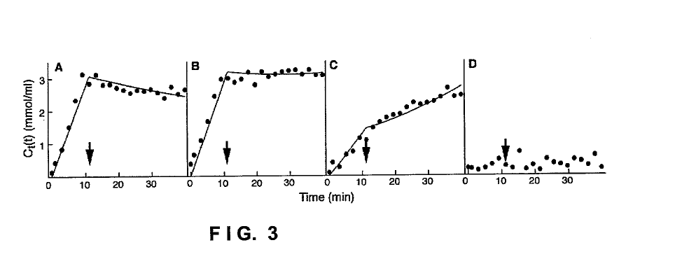

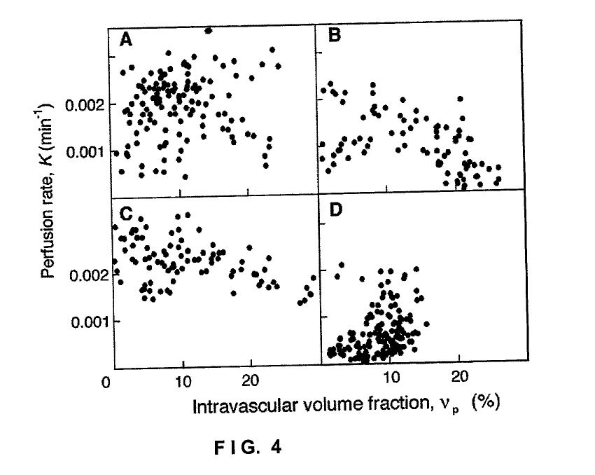

The effect of the perfusion parameters, and vₚ on HDO time evolution per voxel was determined as follows. The rate of HDO uptake during ²H-saline infusion varied depending on the magnitude of the intravascular volume fraction—vₚ. HDO uptake rate was fast in voxels with relatively high vₚ, (FIGS. 4a-b), and was accumulated slowly in voxels with lower vₚ, see FIG. 3C. In such voxels, due to the low intravascular content, the concentration of HDO was relatively low at the end of the infusion, and thus, HDO was not cleared but rather kept accumulating for 30 min. post infusion. In voxels of high vₚ values, the pattern of HDO clearance exhibited either a slow decay (FIG. 4a) or remained apparently constant until the end of the measurements (up to 30 min. after termination of the infusion, see FIG. 3b. In the former case, HDO time course in the voxels was parallel to HDO time course in the blood (FIG. 1a) suggesting that perfusion in such voxels was dominated by the global blood flow. This observation is in accord with the common knowledge that the rate constants of water perfusion across the capillary wall, given by Eq. [3], represent directly the flow since the extraction fraction is approximately one (20). One may therefore deduce that in voxels with high vₚ and no apparent decay of HDO post infusion, such as shown in FIG. 4b, capillary permeability also contributes to the magnitude of the perfusion rate constants. In a few voxels, at the various tumors, ²H₂O concentration remained within noise levels throughout the experimental time, indicating the lack of vascularity in the corresponding tissue (FIG. 3d).

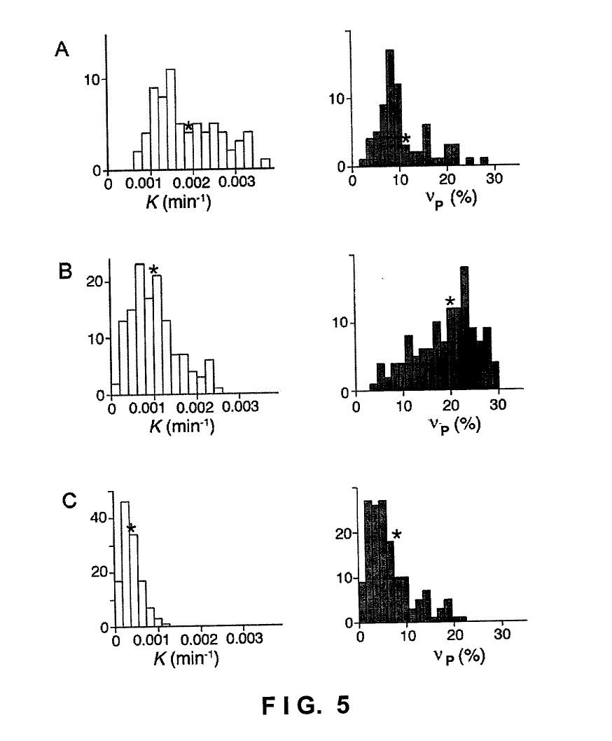

An examination of the correlation between kb and vp revealed the following. Blood cell velocity was shown to correlate linearly with vessel diameter in normal tissues, however, no correlation was found between these two parameters in human glioma (30). Blood cells velocity and vessel diameter have physiological resemblance to the perfusion rate constants and intravascular volume fraction that were measured herein. Therefore, the relation between the perfusion parameters in MCF7 tumors was examined. The general pattern of the K versus vₚ curves varied between the tumor slices and exhibited no specific correlation between the two parameters (FIG. 4). In some tumors the relation between K and vₚ was totally chaotic (FIG. 4a), where in other tumors, a positive or inverse correlation between K and vp was observed in part of the voxels (e.g. FIGS. 4b-d). Also detected were tumors with a small range of low K values and a wide range of increasing vₚ (FIG. 5e), although in most tumor-slices voxels with high vₚ, above 8%, exhibited varying K values including low values.

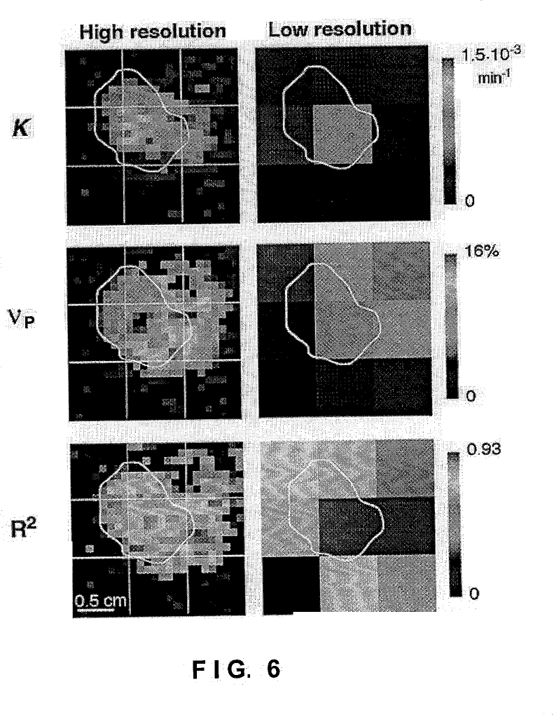

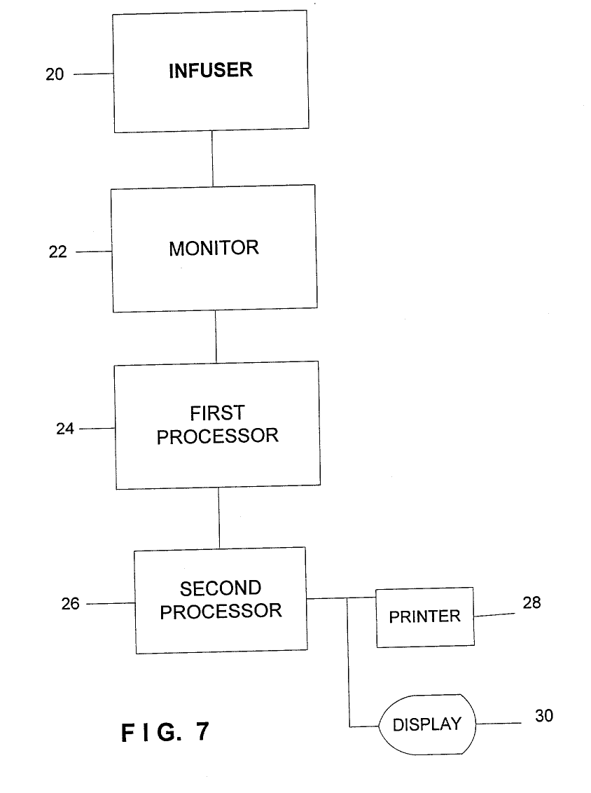

With reference to statistical analysis, the perfusion parameters spanned a wide range of values in the parametric maps, which varied from one tumor to the other, and between slices of the same tumor, as demonstrated by the representative maps in FIG. 3, and summarized for all tumors in Table 1, see FIG. 7. The large variation of the data was also reflected by the high coefficient of variation (c.v.; (43)) per tumor slice (Table 1), which ranged within 37-78% and 18-72% for k_{b} and vₚ, respectively. The distribution of the perfusion parameters in each tumor slice was asymmetric, as shown in FIG. 5. Consequently, the mean values for each perfusion parameter and tumor slice poorly represented the data. The pattern of asymmetry also varied between the tumor slices, representing skewed histograms towards both parts of the K or vₚ scales (e.g. FIG. 6).

The range of the perfusion parameters and its variation about the mean, given by c.v, was tested regarding tumor size. Perfusion was monitored within small tumors, up to 0.5 cm³ of total volume and large tumors, see Table 1.

TABLE 1
Statistical analysis of the perfusion rate and the intravascular
volume fraction, per pixel, in orthotopic MCF7 tumors.

| | | Perfusion rate, K | | Intravascular | |
|---|---|---|---|---|---|
| | size^{b} | (·10⁻³ min⁻¹) | | volume fraction, Vₚ (%) | |
| Index^{a} | (cm³) | Mean | Entire^{c} | Mean | Entire^{c} |
| 1a | 0.21 | 0.56 ± 0.04 | 0.40 | 16.9 ± 0.57 | 12.1 |
| 1b | | 0.64 ± 0.11 | 0.66 | 18.7 ± 1.23 | 19.6 |
| 2a | 0.27 | 1.15 ± 0.07 | 0.68 | 20.7 ± 0.44 | 22.1 |
| 2b | | 1.24 ± 0.09 | 1.20 | 21.9 ± 0.53 | 23.4 |
| 3a | 0.32 | 1.87 ± 0.09 | 1.25 | 10.7 ± 0.65 | 8.0 |
| 4a | 0.54 | 1.15 ± 0.1 | 1.05 | 11.6 ± 0.58 | 12.5 |
| 4b | | 1.43 ± 0.08 | 1.71 | 10.4 ± 0.65 | 11.6 |
| 5a | 0.88 | 0.26 ± 0.02 | 0.22 | 7.13 ± 0.20 | 6.1 |
| 5b | | 0.72 ± 0.03 | 0.77 | 9.29 ± 0.29 | 10.5 |
| 6a | 1.50 | 1.03 ± 0.05 | 0.99 | 19.5 ± 0.58 | 17.7 |
| 6b | | 1.74 ± 0.07 | 1.98 | 22.8 ± 0.49 | 21.1 |
| 7a | 1.79 | 0.40 ± 0.02 | 0.23 | 6.98 ± 0.31 | 9.0 |
| 7b | | 0.88 ± 0.03 | 0.70 | 6.60 ± 0.39 | 9.1 |
| Average^{d} | 0.8 ± | 1.02 ± 0.02 | 0.91 ± | 14.2 ± 0.22 | 14.1 ± 1.6 |
| | 0.2 | | 0.15 | | |

^{a}The indices, a and b, refer to different slices in the same tumor
^{b}Tumor volume was calculated from ¹H-MR images recorded over the whole tumor.
^{c}Entire - the values at a single pixel, including most of the tumor region-of-interest (ROI), in the spatially degraded maps. No significant differences were found between the Mean and Entire values of K (p > 0.1) and vₚ (p > 0.9).
^{d}The average values of the total population of voxels in the ROIs of all tumors (n = 1278 pixels).

The average values and the corresponding standard deviations of the perfusion parameters were similar between the two groups of tumors suggesting that these parameters were independent of tumor size.

A comparison of model-based analysis at low resolution with analysis at high resolution revealed the following. In light of the heterogeneity of the perfusion parameters that were obtained and in order to stress the importance of processing the data at high spatial resolution, the model-based analysis on dynamic ²H-images was applied with a lowered in-plane resolution of 8×16 mm² per voxel (FIG. 6). Those images were produced from the original ²H-MRI data by reducing its in-plane resolution by a factor of 8. In the spatially degraded images, each pixel includes 64 pixels from the high-resolution maps, as is schematically emphasized by the grid layer on the high-resolution maps in FIG. 6. In the spatially degraded maps each tumor slice was represented by 1-2 voxels, as shown by the tumor-slice ROI that is outlined in white in the maps. The corresponding global values of K and vₚ represent a single voxel or the average of 2 voxels per tumor slice (Table 1). The major effect of applying the analysis at low resolution is the loss of ‘hot spots’, namely, the perfusion information is averaged so that very low and very high values disappear from the maps. Specifically, in the analysis that is represented in FIG. 6, the high K values that appear red within the tumor ROI (region of interest) in the high resolution map are not shown in the spatially degraded k_{b} map. In addition, the low vₚ values at the center of the tumor ROI, represented by blue pixels in the high resolution vₚ map (vₚ≦5%), are also averaged in the analysis of the spatially degraded data resulting in a global higher vₚ values at the tumor ROI of the spatially degraded vₚ map. In both cases although it is clear that information is lost, the fitting of the central pixel of the spatially degraded maps, that includes mainly the tumor ROI, is high (R²=0.92), giving the impression that the values behind the fitting are meaningful. However, as was demonstrated in FIG. 5 and discussed previously, the mean values of the high-resolution data as well as the values obtained by the analysis of the degraded resolution, poorly reflect the actual heterogeneity of the perfusion parameters.

Summarizing the foregoing, perfusion parameters were determined in orthotopic planted MCF7 human breast cancer tumors by a model-based analysis of dynamic 3-dimensional ²H-MRI. The experimental model included flow in the intravascular compartment and exchange with the extravascular compartment. Application of kinetic algorithms yielded parametric maps of the perfusion rate , and the intravascular volume fraction, per voxel, which span ranges of 4.0·10⁻⁶-3.9·10⁻³⁻min⁻¹, and 0.4-35%, respectively. The wide range of these parameters was distributed in most tumor slices inhomogeneously around the corresponding mean. The parametric maps displayed heterogeneity of the perfusion parameters within each tumor slice and between one tumor to the other. Analysis of the relation between the rate constants and the corresponding intravascular volume fraction, per voxel, indicated that these two parameters do not correlate in most of the voxels. The heterogeneity of the perfusion parameters together with their variation were completely masked when the data was analyzed at spatially degraded resolution. The later analysis highlighted the importance of monitoring and processing perfusion at high spatial resolution.

Referring now to the apparatus of the invention, the algorithm described above is run on a general purpose computer, such as workstation, which has been appropriately programmed. The programming of the computer is readily accomplished by persons of ordinary skill base on the description of the algorithm as set forth above and the detailed description of the invention. Alternatively, a special purpose computer can be constructed by persons of ordinary skill placing the algorithm as a fixed program in the computer. The computer is fitted with the usual microprocessor, input/output, memory, and display. The computer is connected or coupled to a scanner that generates images, a standard known machine in the art, to receive the data generated by the scanner in the normal course of its operation and functioning. A printer or special display can be included as part of the apparatus, particularly, a color printer, or the output from the computer can be in the form of a storage member, such as a magnetic tape or disc or an optical member, such as a CD Rom or laser disc.

As noted above, the novel algorithm is programmed into the processor of the computer or other processor, and algorithm based processing is effected of the dynamic images obtained from the scanning component, to qualitatively determine tissue perfusion per voxel wherein the algorithm is based on the following equations:

i. during tracer's infusion, t=0−tinfus:

$${C}_{v} ( t ) = {v}_{e} * ( a \cdot t - ( \frac{a}{{k}_{b}} - b ) ( 1 - {e}^{- {k}_{b}^{t}} ) ) + f ( t ) {v}_{p} = {v}_{e}^{*} \cdot {C}_{e} ( {t}_{infus} ) + f ( t ) {v}_{p}$$

ii. after tracer's infusion, t′=tinfus−tend:

$${C}_{v} ( t ) = {v}_{e} * ( c \cdot ( {t}^{′} \cdot {ⅇ}^{- {k}_{b}^{{t}^{′}}} ) - ( \frac{c}{{k}_{b}} - d ) ( 1 - {ⅇ}^{- {k}_{b}^{t}} ) + {C}_{e} ( {t}_{infus} ) {ⅇ}^{- {k}_{b}^{t}} ) = g ( t ) {v}_{p}$$

wherein ve* is the effective volume fraction of the extravascular compartment, vp is the volume fraction of the intravascular (plasma) compartment, Cₑ is tracer's concentration in the extravascular compartment (mmol/ml), Cₚ is tracer's concentration in the intravascular compartment (mmol/ml), Cᵥ is tracer's concentration in a given voxel (mmol/ml) and Cᵥ=Cₑ+Cₚ, kt is rate constant of transfer from vₚ to vₑ*(min−1), k_{b} is rate constant of backflux from vₑ* to vₚ(min−1), a is the rate of tracer accumulation in the intravascular (plasma) compartment, b is the concentration of the tracer in the intravascular (plasma) compartment at the beginning of the tracer's infusion, f(t) is a linear function describing the accumulation of the tracer intravascular (plasma) compartment given by a+bt, c is the washout rate of the tracer from the intravascular (plasma) compartment, d is the concentration of the tracer at the end of its infusion in the intravascular (plasma) compartment and g(t) is a linear function describing the washout of the tracer from the intravascular (plasma) compartment and is given by c+dt.

A second processor is provided from which maps of the perfusion parameters are obtained from the group of: k_{b}, kₜ, vₑ*, vₚ or K where K is perfusion rate (min−1) given by: K=k_{b}×vₑ*, to indicate spatial distribution of tumor perfusion.

In addition to the above, the algorithm can be embedded or stored on a machine readable medium, for example, on a magnetic readable medium like a tape or disc, or on an optically readable medium line a CD Rom or laser disc. In this fashion the medium is a novel product, and is contemplated as part of the invention.

As noted above, the apparatus includes a device that monitors the tracer concentration in the tissue before enrichment, during enrichment and post enrichment by imaging techniques. The device used in the invention is a scanner. The scanner can be selected from among the following types of scanners: an MRI scanner, an optical imaging scanner, a computed tomography (CT) scanner, an ultrasound scanner or a positron emission tomography (PET) scanner. The purpose of the scanner is to obtain dynamic images that will be transferred or transmitted to the processor by a suitable connection or coupling. It is understood that the images may first be stored in a suitable memory that can be part of the scanner, or more preferably, the memory of the computer. The output from the processing, the parametric maps, is stored in the memory, and is also, transmitted to a display where the output, in the form of parametric maps or images, color coded as explained earlier, are available for visual inspection and study. Alternatively or in addition, the output can be sent to a printer that prints the output for visual inspection and study. The output can be sent to a transmitter (this can occur within the computer having an Internet connection) where it can be uploaded to the Internet for transmission to a remote location, at which location it can be downloaded and displayed and/or printed.

In a more specific form of the invention apparatus is provided for monitoring tissue perfusion in a patient that comprising a device, such as an infuser, to infusing into the patient via the patient's bloodstream a substance characterized by being detectable, non-toxic at the concentrations required for detection, transferable through membranes and having the same or similar flow rate as blood flow; an imaging equipment, such as a scanner, for monitoring the concentration of the infused substance in a tissue of interest, before infusion, during infusion and post infusion adjusted to obtain dynamic images; a first processor for algorithm based processing of the obtained dynamic images, to quantitatively determine tissue perfusion per voxel wherein the algorithm is based on the following equations:

i. during tracer's infusion, t=0−tinfus:

$${C}_{v} ( t ) = {v}_{e}^{*} ( a \cdot t - ( \frac{a}{{k}_{b}} - b ) ( 1 - {ⅇ}^{- {k}_{b}^{t}} ) ) + f ( t ) {v}_{p} = {v}_{e}^{*} {C}_{e} ( {t}_{inf us} ) + f ( t ) {v}_{p}$$

ii. after tracer's infusion, t′=tinfus−tend:

$${C}_{v} ( t ) = {v}_{e}^{*} ( c \cdot ( {t}^{′} \cdot {ⅇ}^{- {k}_{b}^{{t}^{′}}} ) - ( \frac{c}{{k}_{b}} - d ) ( 1 - {ⅇ}^{- {k}_{b}^{{t}^{′}}} ) + {C}_{e} ( {t}_{inf us} ) {ⅇ}^{- {k}_{b}^{{t}^{′}}} ) = g ( t ) {v}_{p}$$

wherein vₑ* is the effective volume fraction of the extravascular compartment, vₚ is the volume fraction of the intravascular (plasma) compartment, Cₑ is tracer's concentration in the extravascular compartment (mmol/ml), Cₚ is tracer's concentration in the intravascular compartment (mmol/ml), Cᵥ is tracer's concentration in a given voxel (mmol/ml) and Cᵥ=Cₑ+Cₚ, kₜ is rate constant of transfer from vₚ to vₑ*(min−1), k_{b} is rate constant of backflux from vₑ* to vₚ(min−1), a is the rate of tracer accumulation in the intravascular (plasma) compartment, b is the concentration of the tracer in the intravascular (plasma) compartment at the beginning of the tracer's infusion, f(t) is a linear function describing the accumulation of the tracer intravascular (plasma) compartment given by a+bt, c is the washout rate of the tracer from the intravascular (plasma) compartment, d is the concentration of the tracer at the end of its infusion in the intravascular (plasma) compartment and g(t) is a linear function describing the washout of the tracer from the intravascular (plasma) compartment and is given by c+dt; and a second processor to obtain maps of the perfusion parameters from the group of: k_{b}, kₜ, vₑ*, vₚ or K where K is perfusion rate (min−1) given by: K=k_{b}×vₑ*, to indicate spatial distribution of tumor perfusion.

A flowchart of the apparatus is shown in FIG. 7. As shown, an infuser 20 enriches tissue as described above, and a monitor 22 in the form of a scanner monitors the enrichment as described. The images generated in the monitor are fed to the first processor 24 where the algorithm is run as described, and the results are fed to the second processor 26 where the maps are generated as described. The maps or images are either fed to a printer 28 and/or to a display 30. A memory, not shown, is contained in the processor 24 and/or 28.

Although the invention has been shown and described with respect to a preferred embodiment, nevertheless changes and modifications are possible which do not depart from the teachings herein. Such changes and modifications that are apparent to those skilled in the art from the invention as disclosed herein and the teachings herein are deemed to fall within the purview of the appended claims.

Cited References

1. Jain R K Determinants of tumor blood flow: a review. Cancer Res 1988;48:2641-2658.

2. Shah-Yukich A A and Nelson A C Characterization of solid tumor microvasculature: a three-dimensional analysis using the polymer casting technique. Lab Invest 1988;58:236-244.

3. Less J R, Skalak T C, Sevick E M and Jain R K Microvascular architecture in a mammary carcinoma: branching patterns and vessel dimensions. Cancer Res 1991;51:265-273.

4. Baish J W and Jain R K Fractals and cancer. Cancer Res 2000;60:3683-3688.

5. Jain R K Delivery of molecular and cellular medicine to solid tumors. Adv Drug Deliv Rev 2001;46:149-168.

6. Denekamp J, Dasu A and Waites A Vasculature and microenvironmental gradients: the missing links in novel approaches to cancer therapy? Adv Enzyme Regul 1998;38:281-299.

7. Los M and Voest E E The potential role of antivascular therapy in the adjuvant and neoadjuvant treatment of cancer. Semin Oncol 2001;28:93-105.

8. Beecken W D, Fernandez A, Joussen A M, Achilles E G, Flynn E, Lo K M, Gillies S D, Javaherian K, Folkman J and Shing Y Effect of Antiangiogenic Therapy on Slowly Growing, Poorly Vascularized Tumors in Mice. J Natl Cancer Inst 2001;93:382-387.

9. Furman-Haran E, Margalit R, Grobgeld D and Degani H Dynamic contrast-enhanced magnetic resonance imaging reveals stress-induced angiogenesis in MCF7 human breast tumors. Proc Natl Acad Sci USA 1996;93:6247-6251.

10. Degani H, Gusis V, Weinstein D, Fields S and Strano S Mapping pathophysiological features of breast tumors by MRI at high spatial resolution. Nature Med 1997;3:780-782.

11. Furman-Haran E, Grobgeld D, Margalit R and Degani H Response of MCF7 human breast cancer to tamoxifen: Evaluation by the Three-Time-Point, contrast-enhanced magnetic resonance imaging method. Clinic Cancer Res 1998;4:2299-2304.

12. Tofts P S Modeling tracer kinetics in dynamic Gd-DTPA MR imaging. J Magn Reson Imaging 1997;7:91-101.

13. Barbour H G The basis of the pharmacological action of heavy water in mammals. Yale J Biol Med 1937;9:551-565.

14. Katz J F, Crespi H L, Czajka D M and Finkel A J Course of deuteriation and some physiological effects of deuterium in mice. Am J Physiol 1962;203:907-913.

15. Ackerman J J H, Ewy C S, Becker N N and Shalwitz R A Deuterium nuclear magnetic resonance measurements of blood flow and tissue perfusion employing ²H₂O as a freely diffusible tracer. Proc Nat Acad Sci USA 1987;84:4099-4102.

16. Kim S G and Ackerman J J H Quantitative determination of tumor blood flow and perfusion via deuterium nuclear magnetic resonance spectroscopy in mice. Cancer Res 1988;48:3449-3453.

17. Kim S G and Ackerman J J H Multicompartment analysis of blood flow and tissue perfusion employing D₂O as a freely diffusible tracer: a novel deuterium NMR technique demonstrated via application with murine RIF-1 tumors. Magn Reson Med 1988;8:410426.

18. Mattiello J and Evelhoch J L Relative volume-average murine tumor blood flow measurement via deuterium nuclear magnetic resonance spectroscopy. Magn Reson Med 1991;18:320-334.

19. Neil J J, Song S K and Ackerman J J H Concurrent quantification of tissue metabolism and blood flow via ²H/³¹ P NMR in vivo. Validation of the deuterium NMR washout method for measuring organ perfusion. Magn Reson Med 1992;25:56-66.

20. Kovar D A, Lewis M Z, River J N, Lipton M J and Karczmar G S In vivo imaging of extraction fraction of low molecular weight MR contrast agents and perfusion rate in rodent tumors. Magn Reson Med 1997;38:259-268.

21. Simpson N E and Evelhoch J L Deuterium NMR tissue perfusion measurements using the tracer uptake approach: II. Comparison with microspheres in tumors. Magn Reson Med 1999;42:240-247.

22. Larcombe-McDouall J B and Evelhoch J L Deuterium nuclear magnetic resonance imaging of tracer distribution in D₂O clearance measurements of tumor blood flow in mice. Cancer Res 1990;50:363-369.

23. Eskey C J, Koretsky A P, Domach M M and Jain R K ²H-nuclear magnetic resonance imaging of tumor blood flow: Spatial and temporal heterogeneity in tissue-isolated mammary adenocarcinoma. Cancer Res 1992;52:6010-6019.

24. Eskey C J, Wolmark N, McDowell C L, Domach M M and Jain R K Residence time distributions of various tracers in tumors: implications for drug delivery and blood flow measurement. J Natl Cancer Inst 1994;86:293-299.

25. Kety S S The theory and application of the exchange of inert gas at the lungs and tissues. Pharmacol Rev 1951;3:1-41.

26. Larcombe-McDouall J B, Mattiello J, McCoy C L, Simpson N E, Seyedsadr M and Evelhoch J L Size dependence of regional blood flow in murine turnours using deuterium magnetic resonance imaging. Int J Radiat Biol 1991;60:109-113.

27. Smits G A, Cornel E B, van de Boogert E, Oosterhof G O, Debruyne F M, Schalken J A and Heerschap A Effects of high energy shock waves on tumor blood flow and metabolism: 31P/1H/2H nuclear magnetic resonance study. NMR Biomed 1994;7:319-326.

28. van der Sanden B P J, in't Zandt H J A, Hoofd L, de Graaf R A, Nicolay K, Rijken P F J, van der Kogel A J and Heerschap A Global HDO uptake in human glioma xenografts is related to the perfused capillary distribution. Magn Reson Med 1999;42:479-489.

29. Bentzen L, Horsman M R, Daugaard P and Maxwell R J Non-invasive tumour blood perfusion measurement by ²H magnetic resonance imaging. NMR Biomed 2000; 13:429-437.

30. Yuan F, Salehi H A, Boucher Y, Vasthare U S, Tuma R F and Jain R K Vascular permeability and microcirculation of gliomas and mammary carcinomas transplanted in rat and mouse cranial windows. Cancer Res 1994;54:4564-4568.

31. Patlak C S, Blasberg R G and Fenstermacher J D Graphical evaluation of blood-to-brain transfer constants from multiple-time uptake data. J Cereb Blood Flow Metab 1983;3:1-7.

32. Patlak C S and Blasberg R G Graphical evaluation of blood-to-brain transfer constants from multiple-time uptake data. Generalizations. J Cereb Blood Flow Metab 1985;5:584-590.

33. Maier C F, Paran Y, Bendel P, Rutt B K and Degani H Quantitative diffusion imaging in implanted human breast tumors. Magn Reson Med 1997;37:576-581.

34. Hennig J, Naureth A and Friedburg H RARE imaging: a fast imaging method for clinical MR. Magn Reson Med 1986;3:823-833.

35. Teukolsky S A, Vetterling W T and Flannery B P. Numerical Recipes in C. Cambridge UK: Cambridge Univ. Press; 1994. p 683.

36. Edelstein W A, Glover G H, Hardy C J and Redington R W The intrinsic signal-to-noise fation in NMR imaging. Magn Reson Med 1986;3:604-618.

37. Stanisz G J, Szafer A, Wright G A and Henkelman R M An analytical model of restricted diffusion in bovine optic nerve. Magn Reson Med 1997;37:103-111.

38. Pfeuffer J, Dreher W, Sykova E and Leibfritz D Water signal attenuation in diffusion-weighted 1H NMR experiments during cerebral ischemia: influence of intracellular restrictions, extracellular tortuosity, and exchange. Magn Reson Imaging 1998;16:1023-1032.

39. Pfeuffer J, Flogel U, Dreher W and Leibfritz D Restricted diffusion and exchange of intracellular water: theoretical modeling and diffusion time dependence of 1H NMR measurements on perfused glial cells. NMR Biomed 1998; 11:19-31.

40. Ewy C S, Babcock E E and Ackerman J J H Deuterium nuclear magnetic resonance spin-imaging of D₂O : A potential exogenous MRI label. Magn Reson Imaging 1986;4:407-411.

41. Yeung W T, Lee T Y, Del Maestro R F, Kozak R and Brown T In vivo CT measurement of blood-brain transfer constant of iopamidol in human brain tumors. J Neurooncol 1992;14:177-187.

42. Graham M M Physiologic smoothing of blood time-activity curves for PET data analysis. J Nucl Med 1997;38:1161-1168.

43. Ott R L and Longnecker M. An introduction to statistical methods and data analysis. Pacific Grove, Calif.: Duxbury; 2001.

44. Furman-Haran E, Grobgeld D, Kelcz F and Degani H Critical role of spatial resolution in dynamic contrast-enhanced breast MRI. J Magn Reson Imaging 2001;13:862-867.

45. Simpson N E, He Z and Evelhoch J L Deuterium NMR tissue perfusion measurements using the tracer uptake approach: I. Optimization of methods. Magn Reson Med 1999;42:42-52.

46. Furman-Haran E, Grobgeld D and Degani H Dynamic contrast enhanced imaging and analysis at high spatial resolution of MCF7 human breast tumors. J Magn Reson 1997; 128:161-171.

What is claimed is:

1. A method for monitoring tissue perfusion comprising the steps of:
a. enriching a living tissue mass, including a tumor, with a tracer characterized by being a small molecule, detectable, non-toxic at the concentrations required for detection, and having a flow rate similar or faster than that of the blood flow;
b. monitoring the tracer concentration in the tissue before enrichment, during enrichment and post enrichment by an imaging technique selected from the group consisting of MRI, optical imaging, computed tomography (CT), ultrasound or positron emission tomography (PET), to obtain dynamic images;
c. processing the obtained images to quantitatively determine perfusion per voxel of the images; and
d. obtaining maps of perfusion parameters to indicate spatial distribution of the tissue perfusion.

2. The method of claim 1 wherein the tracer is selected from the group consisting of: water and water labeled with deuterium, or tritium, or ¹⁷O labeled water (H₂¹⁷O), or ¹⁸O labeled water, sugars including mannitol and sugars including mannitol labeled with ¹⁴C, ¹³C or ²H or ³H or ¹⁷O or ¹⁸O, alcohols including ethanol and alcohols labeled with ¹⁴C, ¹³C or ²H or ³H or ¹⁷O or ¹⁸O, organic acids including acetic and lactic acid and organic acids with ¹⁴C, ¹³C or ²H or ³H or ¹⁷O or ¹⁸O, amines including ethanolamine, amino acids and analogs of amino acids and amines with ¹⁵N, ¹⁴C, ¹³C or ²H or ³H or ¹⁷O or ¹⁸O, small fluorinated compounds with either ¹⁹F or ¹⁸F and glucose labeled with ¹³C or ²H or ³H.

3. The method of claim 1 wherein perfusion parameters include intravascular volume fraction of the tissue, perfusion rate constants or perfusion rate.

4. The method of claim 1 including the further step of generating a proportion of variability map.

5. The method of claim 1 wherein the enriching step is carried out using deuterated-saline solution.

6. The method of claim 1 wherein the enriching step is carried out by intravenous infusion of a deuterated-saline solution to obtain high and non-toxic levels of HDO in the blood.

7. The method of claim 1 wherein the images are processed at voxel resolution.

8. The method of claim 7 wherein the voxel size is less than about 0.01 cm³.

9. The method of claim 1 wherein the processing step is carried out using a pair of equations, one for time 0 to end of infusion and one for time end of infusion to end of magnetic resonance imaging, and best fitting obtained image data at a voxel resolution.

10. The method of claim 9 wherein the best fit is greater than 0.7 in a major fraction of the voxels.

11. The method of claim 10 including the further step of generating a proportion of variability map.

12. The method of claim 1 wherein the generated maps are portrayed in color code.

13. The method of claim 1 wherein the generated maps are displayed.

14. A method for monitoring a patient having a tumor to determine perfusion tumor heterogeneity comprising the steps of:
a, Infusing into the patient via the patient's bloodstream a substance characterized by being detectable, non-toxic at the concentrations required for detection, having a flow rate similar or faster than that of the blood flow;
b. monitoring the tumor before infusion, during infusion and post infusion to obtain dynamic ²H magnetic resonance images with predetermined high spatial resolution;
c. processing the obtained images to quantitatively determine perfusion per voxel of the images; and
d. obtaining maps of perfusion parameters to indicate spatial distribution of the tumor perfusion.

15. The method of claim 14 wherein perfusion parameters include intravascular volume fraction of the tissue, perfusion rate constants or perfusion rate.

16. The method of claim 14 wherein the tracer is one of the following: deuterated labeled water, water and water labeled with tritium, or ¹⁷O labeled water (H₂¹⁷O), or ¹⁸O labeled water, sugars including mannitol and sugars including mannitol labeled with ¹⁴C, ¹³C or ²H or ³H or ¹⁷O or ¹⁸O, alcohols including ethanol and alcohols labeled with ¹⁴C, ¹³C or ²H or ³H or ¹⁷O or ¹⁸O, organic acids including acetic and lactic acid and organic acids with ¹⁴C, ¹³C or ²H or ³H or ¹⁷O or ¹⁸O, amines including ethanolamine, amino acids and analogs of amino acids and amines with ¹⁵N, ¹⁴C, ¹³C or ²H or ³H or ¹⁷O or ¹⁸O and small fluorinated compounds with either ¹⁹F or ¹⁸F, glucose labeled with ¹³C or ²H or ³H.

17. The method of claim 14 including the further step of generating a proportion of variability map.

18. The method of claim 14 wherein the enriching step is carried out using deuterated-saline solution.

19. The method of claim 14 wherein the enriching step is carried out by infusing into the living tissue mass containing blood capillaries a deuterated-saline solution to obtain high and non-toxic levels of HDO in the blood.

20. The method of claim 14 wherein the images are processed at voxel resolution.

21. The method of claim 20 wherein the voxel size is less than about 0.01 cm³.

22. The method of claim 14 wherein the processing step is carried out using a pair of equations, one for time 0 to end of infusion and one for time end of infusion to end of magnetic resonance imaging, and best fitting obtained image data at a voxel resolution.

23. The method of claim 22 wherein the best fit is greater than 0.7 in a major fraction of the voxels.

24. The method of claim 22 including the further step of generating a proportion of variability map.

25. The method of claim 14 wherein the generated maps are portrayed in color code.

26. The method of claim 14 wherein the generated maps are displayed.

27. Apparatus for monitoring tissue perfusion in a patient comprising:
a. a device to infusing into the patient via the patient's bloodstream a substance characterized by being detectable, non-toxic at the concentrations required for detection, transferable through membranes and having the same or similar flow rate as blood flow;
b. imaging equipment for monitoring the concentration of said substance in a tissue of interest before infusion, during infusion and post infusion adjusted to obtain dynamic images;
c. a first processor for algorithm based processing of the obtained dynamic images, to quantitatively determine tissue perfusion per voxel wherein the algorithm is based on the following equations:
i. during tracer's infusion, t=0−tinfus:
$${C}_{v} ( t ) = {v}_{e}^{*} ( a \cdot t - ( \frac{a}{{k}_{b}} - b ) ( 1 - {ⅇ}^{- {k}_{b}^{t}} ) ) + f ( t ) {v}_{p} = {v}_{e}^{*} {C}_{e} ( {t}_{inf us} ) + f ( t ) {v}_{p}$$
ii. after tracer's infusion, t′=t_{infus}−t_{end}:
$${C}_{v} ( t ) = {v}_{e}^{*} ( c \cdot ( {t}^{′} \cdot {ⅇ}^{- {k}_{b}^{{t}^{′}}} ) - ( \frac{c}{{k}_{b}} - d ) ( 1 - {ⅇ}^{- {k}_{b}^{{t}^{′}}} ) + {C}_{e} ( {t}_{inf us} ) {ⅇ}^{- {k}_{b}^{{t}^{′}}} ) = g ( t ) {v}_{p}$$
wherein vₑ* is the effective volume fraction of the extravascular compartment, vₚ is the volume fraction of the intravascular (plasma) compartment, Cₑ is tracer's concentration in the extravascular compartment (mmol/ml), Cₚ is tracer's concentration in the intravascular compartment (mmol/ml), Cᵥ is tracer's concentration in a given voxel (mmol/ml) and Cᵥ=Cₑ+Cₚ, kₜ is rate constant of transfer from vₚ to vₑ* (min⁻¹), k_{b} is rate constant of backflux from vₑ* to vₚ(min⁻¹), a is the rate of tracer accumulation in the intravascular (plasma) compartment, b is the concentration of the tracer in the intravascular (plasma) compartment at the beginning of the tracer's infusion, f(t) is a linear function describing the accumulation of the tracer intravascular (plasma) compartment given by a+bt, c is the washout rate of the tracer from the intravascular (plasma) compartment, d is the concentration of the tracer at the end of its infusion in the intravascular (plasma) compartment and g(t) is a linear function describing the washout of the tracer from the intravascular (plasma) compartment and is given by c+dt; and
d. a second processor to obtain maps of perfusion parameters from the group of: k_{b}, kₜ, vₑ*, vₚ or K where K is perfusion rate (min⁻¹) given by: K=k_{b}×vₑ*, to indicate spatial distribution of tumor perfusion.

28. Apparatus for monitoring tissue perfusion in a patient comprising:
a. an infuser to infuse into the patient via the patient's bloodstream a substance characterized by being detectable, non-toxic at the concentrations required for detection, transferable through membranes and having the same or similar flow rate as blood flow;
b. an imager for monitoring the concentration of said substance in a tissue of interest before infusion, during infusion and post infusion adjusted to obtain dynamic images;
c. a first processor for algorithm based processing coupled to receive the obtained dynamic images, to quantitatively determine tissue perfusion per voxel wherein the algorithm uses a pair of equations, one for time 0 to end of infusion and one for time end of infusion to end of imaging, and best fits obtained image data at a voxel resolution;
d. a second processor to obtain maps of perfusion parameters to indicate spatial distribution of tumor perfusion from the determined tissue perfusion per voxel.

29. Apparatus according to claim 28 further including a display for portraying the maps of perfusion parameters.

30. Apparatus according to claim 28 wherein the first processor processes images at voxel resolution.

31. Apparatus according to claim 30 wherein the voxel size is less than about 0.01 cm³.

32. Apparatus according to claim 28 wherein the best fit is greater than 0.7 in a major fraction of the voxels.

33. Apparatus for monitoring a patient having a tumor to determine perfusion tumor heterogeneity comprising:
a, an infuser for infusing into the patient via the patient's bloodstream a substance characterized by being detectable, non-toxic at the concentrations required for detection, having a flow rate similar or faster than that of the blood flow
b. a monitor for monitoring the tumor before infusion, during infusion and post infusion to obtain dynamic ²H magnetic resonance images with predetermined high spatial resolution;
c. a processor for processing the obtained images to quantitatively determine perfusion per voxel of the images; and
d. a second processor to obtain maps of perfusion parameters to indicate spatial distribution of tumor perfusion from the determined tissue perfusion per voxel.
e. a device to portray maps of determined perfusion parameters to indicate spatial distribution of the tumor perfusion.

34. A machine readable medium having stored thereon an algorithm comprising a pair of equations, one for time 0 to end of infusion and one for time end of infusion to end of imaging, and best fitting for obtained image data at a voxel resolution for dynamic images with predetermined spatial resolution obtained from an imaging.
